(19) United States
(12) Patent Application Publication
(10) Pub. No.: US 2016/0235708 A1
(43) Pub. Date: Aug. 18, 2016

(54) TOPICAL PIGMENTORY COMPOSITION

(71) Applicant: Sanjay BANERJI, Kanpur (IN)

(72) Inventors: Sanjay Banerji, Kanpur (IN); Supriya Sen, Kanpur (IN)

(21) Appl. No.: 15/025,175

(22) PCT Filed: Jul. 3, 2014
(86) PCT No.: PCT/IN2014/000443

§ 371 Date: Mar. 25, 2016

(30) Foreign Application Priority Data
Oct. 4, 2013 (IN) 2943/DEL/2013

(51) Int. Cl. A61K 31/37; A61K 9/00; A61K 47/20; A61K 47/10; A61N 5/06; A61K 31/137; A61K 47/22; A61K 45/06; A61K 31/436; A61K 9/08; A61K 41/00; A61K 47/08

(52) U.S. Cl. CPC A61K 31/37; A61K 41/0066; A61K 9/0014; A61K 47/20; A61K 47/10; A61K 47/08; A61K 31/137; A61K 47/22; A61K 45/06; A61K 31/436; A61K 9/08; A61N 5/062; A61N 2005/0657; A61N 2005/0661; A61N 2005/067

(57) ABSTRACT

A composition is provided for topical photochemotherapy for skin diseases, more specifically it comprises of a topical composition comprising of at least one photoactive agent along with detectable marker for treatment of various dermatological conditions including but not limited to Vitiligo, Psoriasis Alopecia Areata and other skin diseases those respond to photochemotherapy.

### FIELD OF INVENTION

This invention relates to topical photochemotherapy for skin diseases, more specifically it comprises of a topical composition comprising of at least one photoactive agent and a colorant or a mixture of colorants thereof which acts as a detectable marker for topical treatment of various Dermatological conditions including but not limited to Vitiligo, Psoriasis and Alopecia Areata. Optionally a corticosteroid or another topically used active ingredient may be included.

### BACKGROUND

To bring out the novelty and usefulness of this invention a brief description of diseases wherein the invention can be used, their present day therapy, limitations and drawbacks of the present day therapeutic measures, related and prior art, how does this invention addresses the drawbacks of presently available therapeutic measures along with various aspects of the present invention are described hereinbelow:

Vitiligo—Vitiligo is an acquired dermatological disease characterized by focal or widespread depigmentation of the skin due to selective loss of melanocyte, the cells responsible for pigmentory activity. The incidence of Vitiligo according to various studies varies from 1-2.00% of population. The disease is of great concern when it affects the colored population because of the contrast it produces in the color of the skin between the areas affected and non affected. Thus the disease carries a strong social stigma amongst the colored population in the Indian subcontinent region in countries like India, Pakisthan, Bangla Desh, Sri Lanka etc. Beside the cosmetic problem, it brings a great agony leading to psychological and social problem because of the believe that the disease is a hereditary and the off springs will be affected by the disease thus not only the affected individual but the whole family faces the matrimonial problem, as an individual not having the disease do not wants to get married to an affected individual. Further it is often believed that the disease is of infective nature as according to Ayurvedic (Indian system of Medicine) the disease has been described as ‘Safed Korh’ and Korh is referred to Leprosy which still remains a dreaded condition amongst the ignorant mass. Clinically Vitiligo can be classified as Localized, generalized and segmental.

In localized Vitiligo small area of the body surface is involved whereas in generalized type a wide spread distribution of lesions are found, in segmental type it has an unilateral distribution with a dermatomal distribution, Vitiligo often involves the mucous membrane and the muco-cutaneous junctions such as lips and genital region, Vitiligo may also affect the hairs and then the condition is referred to as Leucotrichiosis. The course of the disease is unpredictable, it may remain static and localized for long period, it may have slow progression or it may be rapidly progressive, depending on the clinical course the disease process is defined as stationary, progressive, improving or resistant. Because of these factors the patients suffering from the disease seek early and effective treatment.

Treatment—Present day Treatment of Vitiligo consists of Medical, surgical or a combination thereof, beside the cosmetic camouflage.

Medical treatment is the mainstay treatment of vitiligo in all its variety and stages, it includes both topical and systemic, often we have to use both the therapy at same time. The topical agents being used are photoactive or photosensitizing agent Psoralen or Psoralen based compounds, corticosteroids, immunomodulators such as Tacrolimus, Pimecrolimus, these are besides the miscellaneous agents of doubtful value with conflicting claim such as Placental extracts, Melaginina Pseudocatalase, basic Fibrocyte Growth factor.

Topical therapy alone is mostly recommended in cases of localized vitiligo or when the involvement is less than 10% of total body surface area (BSA)—Guidelines for Medical Management of Vitiligo Published by IADVL Pg 11, 2009

Topical treatment alone is also recommended in cases of children, patients over 50 yrs of age and for those who cannot tolerate oral therapy or systemic therapy is contraindicated such as patients with liver disorders. Topical therapy along with UVR is most widely used in India in these cases. Topical Psoralen therapy is also recommended where systemic Psoralen cannot be used because of impaired hepatic function or for those who cannot tolerate oral Psoralen. Topical Psoralen therapy is also given used along with Systemic therapy.

The systemic agents those have been or used includes the Oral Psoralens along with Phototherapy, Systemic corticosteroids, immunomodulators such as Levamisole, Azathiopurine, cyclophosphomide methotrexate, often one of these or in combination are used along with the topical agents.

Surgical treatment—This form of therapy is mostly required and indicated in localized or resistant to medical therapy cases.

Though this invention is in particular useful for treatment of Vitiligo it can be used without limitations in treatment of other dermatological conditions where ever photochemotherapy is indicated such as Psoriasis, Alopecia Areata some type of eczemas.

### Drugs Used in Treatment of Vitiligo

Psoralens—Psoralens have been used in treatment of Vitiligo for long and as of today they remain the sheet anchor in medical management of Vitiligo. Psoralens have been and are used topically, systemically as well as both. They are naturally occurring tricyclic compound present in plants fruits such as lime, figs parsnip, bael, Ammi Majus (Popularly known as babchi or Bokuchi in India, Bael Etc) beside these natural sources of psoralens or Psoralen derivatives. Commonly used Psoralen derivatives are 4, 5, 8 Trimethyl Psoralen, 5, Methoxypsoralen and 8-methoxy psoralen (Methoxsalen) which is both natural or synthetically derived.

4, 5, 8, Tri methyl psoralen is synthetic Psoralen. These have the property of absorbing and transferring the ultra violet rays to the human cells to cause desired therapeutic effect thus they are termed as photoactivable compounds and the changes they produce in the skin is termed as photosensitization thus they are also known as photosensitizing agents, beside 8 methoxypsoralen and 4, 5′ 8 trimethylpsoralen there are other psoralen derivatives such as those described in, U.S. Pat. No. 4,321,919 and U.S. Pat. No. 5,399,719. The photoactivable compounds those can be used in accordance with the present invention include psoralen and psoralen derivatives; such as 4,5′8-trimethylpsoralen; 5-methoxypsoralen; 4-methylpsoralen; 4,4-dimethylpsoralen; 4-5′- dimethylpsoralen; 4′- hydroxymethy 1-4, 5′,8 trimethylpsoralen; 4′,8-methoxypsoralen; and a 4′-(omega-amino-2-oxa) alkyl-4,5′,8-trimethylpsoralen. Presently the widely used photosensitising compound is 8-methoxypsoralen (9-methoxy-7H-furo[3,2-g][1]-benzopyran-7-one or 8-MOP) and 4 5′ 8 Tri methyl psoralen. 8-Methoxysalen is a naturally occurring photoactive substance found in the seeds of the Ammi majus (umbelliferae plant). See, for example, Fahmy et al., “Ammi Majus Linn Pharmacognostical Study and Isolation of Crystalline Constituent, Ammoidia”, Quant. J. Pharm. and Pharmacol., 20:281, Psoralen it's derivatives are not effective unless they are used along with Ultra Violet ray exposure they are to be used along with the Photo element (UVR) and this form of therapy is termed as Photochemotherapy. The most commonly used 8 methoxypsoralen (Methoxsalen) 1% topical solution (USP) is colorless and is not visible on topical application.

Corticisteroids—Way back in 1970 Kandil E, reported the beneficial effect of corticosteroid in treatment of Vitiligo. (Ref. Treatment of localized Vitiligo with intralesional triacinolone acetonide Dermatologica 1970, 140: 195, 1970) Corticosteroids are a group of drugs similar to the hormones produced by the adrenal glands, topical corticosteroids are effective in vitiligo by their anti inflammatory immunosuppressive action. Topically applied corticosteroid are classified according to their property as mild, mid potent and super potent. It is the mid and super potent corticosteroid those are effective in Vitiligo, the mid potent corticosteroids commonly used are Betamehtasone valerate, Betamethasone dipropionate, mometasone furoate fluticasone propionate. Therapeutic use of topical corticosteroid should be monitored and carried out under the supervision of a doctor as it may cause side effects such as atrophy of the skin, telangiectesia, appearance of striae, hyperpigmentation and hypertrichiosis, duration of therapy may extend beyond 3 months. It is the simplest and safest treatment but not as effective as psoralen in form of photochemotherapy. Any topically used corticosteroid may be included in formulating a dossier of the present composition. An incomplete list of topically used corticosteroids include Hydrocortisone Acetate, Halobetasol Hydrocortisone Butyrate, Beclometasone Beclomethasone dipropionate, Betamethasone Valerate, Clobetasone, Clobetasol propinate, Dexamethasone, Fluticasone propionate Flucinolone Acetonide, Mometasone Furoate, Triamcinolone, triamcinolone Acetonide and other corticosteroids, preferably it is the mid potent or potent corticosteroids be used however in children mold corticosteroids are preferable, mild corticosteroid are preferable when treating areas like face axilla groins. The dose of the topical corticosteroid varies with one with the other preferably the doses as mentioned in pharmacopoeias such as USP/BP/IP/NP are used in formulating the dosage.

The choice of the topical corticosteroid in the composition will depend on multiple factors, the inventors choice are of mid potent Varity such as Fluticasone Propinate, Betamethasone Valerate, those can be used once a day application, however the choice of the corticosteroid is not limiting to those mentioned and in this invention is not a binding factor.

PUVA (Photochemotherapy)—This is a form of therapy wherein a photosensitizing drug is used systemically and/or topically followed by Ultra Violet Rays exposure. Psoralen or psoralen based drugs suh as 4,5,8 Trimethyl Psoralen or 8 methoxypsoralen are commonly used drugs for treatment of various dermatosis such as Vitiligo, Psoriasis, Alopecia areata, lchen planus, chronic eczema, Pre cancerous condition such as Mycosis fungoidis, some of these conditions have an Auto immune background others are of unknown etiology.

The source of Ultra violet light being used can be natural sunlight or artificial lamps emitting Ultra violet rays, in case of sunlight it is often referred to as PUVASOL therapy indicative of solar energy. Ultra Violet light refers to electromagnetic spectrum between 290-400 nm .this is divided in UVB having spectrum between 290-320 and UVA having spectrum between 320-400 nm The solar spectrum contains both UVA as well as UVB. Presently both UVA and UVB are used as a source of energy. It is well accepted that orally administered 8-methoxypsoralen followed by irradiation with artificial UV light is effective in treatment of Psoriasis, this is referred to as PUVA Psoralens have a special property of absorbing and transferring various band of spectra in the UV range and passing it to the living cells of human beings causing various changes.

Though UVB alone has mostly being used in treatment of Vitiligo there are stray reports of using UVB along with Psoralen, thus this invention may not limit to use along with UVA but it can be used along with UVB. UVC has also been used occasionally in phototherapy and the photoactive composition of this invention can also be used along with UVC. Other misclaneous Drugs reported to be of beneficial effects in treatment of Vitiligo includes Immuno modulators like Tacrolimus, Fibrocyte growth factor, Placental extract, Melaginina.

DTPC—Detectable Topical Photoactive Composition—In abbreviated form the present invention can be referred to as DTPC., in expanded form it refers to Detectable Topical Photoactive Composition meaning area of skin or mucous membrane wherein the composition has been applied is visible or detectable under ultra violet rays.
Detectable Marker—Detectable Marker in this invention refers to an agent used in this composition which imparts a color and is visible in presence of light and/or under Ultra Violet Rays to the composition or when the it is topically applied. The detectable marker used in this invention preferably pharmaceutically acceptable, non toxic, temporarily staining which can easily be wiped or washed with water or any harmless solvent such as alcohol or acetone. Detectable marker preferably used is a colorant which can be a pigment and/or dye or it's lake as defined in Ramingtons Pharmaceutical Sciences 16 Th Ed 1980 The colorants used maybe those approved for use for external application(External D & C colors), those approved for Food, drugs and cosmetics (F D & C) or D & C (Colors used in Drugs & Cosmetics). The updated list of FDA approved colorants is available in their website and by mention those are included herein in entirety. An incomplete list of colorants those may be used are Patent Blue, brilliant blue, brilliant green, Sunset yellow, Tartrazine, Quinanzarine further it can be a combination of colorants any other regulatory and pharmaceutically acceptable colorants can be used in the invention. Lakes are also known in the art of colorants they are the salts of various dyes the advantage of using these are their solubility in water they can preferably be used in this invention as a colorant.

The regulatory status of colorants widely varies from one country to other as discussed in review article of Krishna vamshi Alam and Gannu Praveen kumar titled Colorants- The cosmetics for the Pharmaceutical dosage Form (International Journal of Pharmacy and Pharmaceutical science Vol 3, suppl 3, 2011 thus preferably the colorant used be an regulatory approved further list of colorants approved by FDA is listed in this review article and by mention all those listed colorants may be used singly or in combination in this invention. Without limitations any coloring agent/agents in combination thereof, pharmacologically acceptable and approved from regulatory angle in the country of produce and marketing can be used in this invention. Stains are also known in the art they colorants used in pathological work for staining the tissue material and Bacteriological work, they are also used for staining of surgical dressing. they are soluble in aqua, Alcohol or any other solvent system an incomplete list of such colorants include Acraflavin, brilliant green fusthin (acid on base) Malachite Green, Indigo Carmine, eosin, Methylene Blue, Florasein Congo red, Gentian Voilet some of these stains in addition to the staining property have antibacterial property such as Acraflavin Brilliant green, They can also be used as or colorant in the present in invention.

In their disclosure US patent application 20100239619 Hurwitz, Marni Markell mentions about topical use of semi permanent color along with the method of application. The dye used as detectable marker in this invention preferably be washable with and should not be of permanent nature. Though it is preferred that the detectable marker is washable with water, however, it may require use of alcohol, acetone, purified water or a combination thereof for removal.

### Major Drawback of Present Day Topical Photochemotherapy:

### Pigmentory Changes Beyond the Area of Topical Application

A very common complication with topical Psoralen and other Psoralen derivatives such as Trimethylpsoralen and 8 methoxypsoralen is phototoxic reaction leading to erythema blistering Hypo/hyper pigmentation beyond the lesion because of various factors such as the spread of the topical composition beyond the margin of the lesion thereby leading to blistering surrounding the lesion or there may be hyperpigmentation surrounding the lesion FIG. 1A shows hyperpigmentation following the use of Methoxsalen, FIG. 1B shows erythematous lesion surrounding the Vitiligo patch following topical PUVA therapy, secondly topical application of Psoralen particularly in lotion base may lead to trickling of the lotion thereby leading to hyperpigmentation along the line of trickling as cited in Melanin Pigmentary Disorder Treatment Update Dermatology Clinics Vol. 23 No. 2 April 2005 Jean-Paul Ortonne Thierry Passeron Pg. 211

Exagerrated Photo Toxic Reaction—This happens due to extra unwarranted UVR exposure particularly on sun exposed areas due to incomplete removal of topical agent because of improper wiping as it is not visible to eye.

### PRIOR ART

To overcome the above mentioned drawbacks of topical Psoralen therapy it is often advised to apply a sunscreen after the desired period of Psoralen and Ultra Violet therapy exposure thus the cost of the therapy goes up because patient has to go on for two formulations. To overcome this drawback of topical photochemotherapy Decola Dennis et. al in their U.S. Patent application No 20060134031 discloses of a composition comprising of a photoactivable agent and an agent that blocks the extraneous radiation during photochemotherapy, however, the combination does not contain the marker, as such on topical application it may go beyond the area of the disease and use of photoactive and photoabsorbing agents may not be liked logical for all, McElenery et. in their U.S. Pat. No. 6,086,858 teaches us the use of colorant along with sunscreen composition based on changing of color on application over the skin as the color used changes pH basis, they have also used fluroscent color along with composition which gets clear on topical application, U.S. Pat. No. 6,214,322 of Castro et al. teaches us of a sun less tanning cosmetic composition comprising of carmine as the colorant along with self tanning agents, this is different from present composition wherein a colorant is being used for detectable purpose and involves the use of Ultra violet rays further in tanning the colorant are semi permanent whereas in the present composition the colorant is washable. U.S. Pat. No. 5,556,612 teaches us the use of photosensitizing agent over the disease area to be treated and the use of photo absorbing agent surrounding the normal skin while treating proliferative skin conditions such as Psoriasis, lichen planus. A very close to this invention is patent application US 20080085245 Sheil; Meridith, Giffard; Allan Olsson; Charles Robert disclose the use of Topical anesthetic composition along with food dye as a detectable marker for vetirnery use, another very close to the present invention is U.S. Pat. No. 7,422,388 of Tufts et al which teaches us use of colored antibacterial composition comprising of the antibacterial agent Chlorohexidine Gluconate and an applicator made of porous element for targeted application. In their patent application 20100119561 Ralph et al discloses about Polymeric particles loaded with dye in composition to indicate the user that the composition has performed it's intended purpose by change of color produced by mixing of the particles by fracturing and fragmentation into smaller pieces during application of the composition through the energy provided by the user as in tooth brushing, however, this is not suitable the present composition as the purpose here is to apply the medicament in a targeted and artistic form without any vigorous rubbing, further it is also suggested in their disclosure that at least two minutes of brushing is required for proper mixing of polymeric particles this is also not possible because in Vitiligo topical application may have to be made at numerous lesions and two minutes of vigorous rubbing at all the areas will involve too much time thus the use of dye loaded polymeric particles is not suitable for the present composition, further the dye loaded polymeric particles is suited for emulsion, gels and dispersion, whereas the dosage form most suitable in the present composition is solution wherein the ingredients are fully dissolved which allows free flow of the composition and no vigorous rubbing is required for fracturing, fragmentation and mixing.

### How Does the Composition Addresses the Drawbacks of Topical Photochemotherapeutic Agents Presently Available.

The composition described herein takes care of the drawbacks stated above by facilitating the restriction of application of the topical composition only over the lesion/lesions or area requiring therapy as one can see the colored area where the application of the composition has been made and any extension of application beyond the area of therapy can be taken care and wiped off immediately before exposing to Ultra violet rays, further avoidance of trickling of the lotion is taken care of as it facilitates targeted application of the composition this allows the patient, attendant or the healthcare personal take care of any spilling or application of the medicament beyond the area of lesion, this invention is particularly useful in cases of Vitligo as many a time lesions are very small in size with irregular borders as depicted in FIG. 2-A & 3-A and it become difficult to restrict the application of topical medicament only to the lesion/lesions.

The composition containing the photoactive agent and a colorant attends to another complication associated with presently available therapeutic formulation is that it is advised with photochemotherapy to wipe of the medicament following UVR exposure, however, many a times one cannot make out complete clearance of the topical agent or forgets to wipe off and this leads to extra unwanted exposure of UVR on going out in the sun, more so in sun exposed area leading to exaggerated photo toxic reaction in form of erythema and blistering due to overexposure, this is taken care by the colored composition as one can easily make out that wiping has been done properly or not. Yet another advantage of present composition is that manufacturers can use different colors for different strengths of topical medicament as some areas of body require lesser concentration of the active ingredient such as face and patient can be guided accordingly.

### DETAILED DESCRIPTION OF THE INVENTION

A detailed description is presented herein which will provide better understanding of the various aspects of the invention which includes the main component of the composition, what can be added, mixed, incorporated in the composition while formulating the therapeutic dossier, how the composition can be used, what are the advantages of the composition compared to existing products etc.

The main components of the composition is a photo active agent, preferably the photo active agent is Psoralen or it's derivatives which can be a natural or synthetically derivative more preferably the psoralen component is Trimethyl Psoralen, 5 Methoxy Psoralen or 8 Methoxypsoralen The other main component of the composition is a colorant or coloring agents which can be a natural or synthetic color or a combination thereof, as defined in chapter on colorants under Pharmaceutical Necessities in Ramingtons Pharmaceutical Sciences 16 th edition 1980.

The colorant used in composition preferably be regulatory approved for use in food, drugs and/or cosmetic in country where it is produced, brands available in India in under the trade name IDACOL, ANUJA. Without limitations any of the colorants can be used many of them are mentioned in patent application No.20100119561 and by mention it is included in entirety, other colorants can also be used depending on their regulatory status. the colorant used should be minimum but adequate enough to leave a temporary mark on the skin.

The strengths of the psoralen component can vary, in case of 8 methoxypsoralen this can vary from 0.001-10% preferably between 0.1-10% more preferably between 0.5-2.0%,still more preferably between 0.5-1.0%,

The use of composition is for the skin condition those responds to photochemo therapy which includes but not limits to Vitiligo, Psoriasis, Alopecia areata. The use of the composition as in photochemo therapy the use of the agent is along with ultra Violet Rays (UVR) the same applies here also the composition is to be used along with ultra violet rays the source of ultra violet rays can be natural sun light known has PUVASOL indicative the use of solar rays as source of ultraviolet rays on an artificial source emitting UVA/UVB. The method of use involves application of the composition followed by exposure to UVR, the time period between application of the composition and exposure may vary and it can be any time within 3 hours of application or later, however it is preferably to be between 10-60 minutes following application of the composition at the site of lesion/lesions, further the exposure time may also vary preferably it is between 30 seconds to 20 minutes, usually the time period of exposure increases with subsequent exposure and threshold is reached to get the desired results, the treatment schedule may vary from once a week exposure to alternate day exposure, further this time period may vary according to the type of skin color of the individual as described by Fitzpatrick classification in chapter on Photodermatosis in Braun Falcos Dermatology Vol. 1, Chapter 41, 3rd Ed., These time period may vary and this does not limits the scope of invention.

In addition to the main components of the composition other agents those can be included in the composition can be various agents those referred to as ‘Pharmaceutical Necessities’ in Chapter 67 Ramingtons Pharmaceutical Sciences !980. Pharmaceutical Necessities are substances those are useful for in preparation and compounding of dosage form, these agents can be Anti oxidants, Buffering agents, carriers diluents, flavoring agents, emulsifying suspending agents, gelling agents, ointment bases, preservatives.

Further other active ingredients those can be included in composition include another active ingredients used in treatment of Vitiligo such as the immune modulator Tacrolimus, Primecrolimus Calcipotriol, Melagenina, Placental extract, Fibrocyte actvating Factor, anti-inflammatory such as corticosteroids, any of the corticosteroid used in topical treatment can be included, it can be a mild, moderately potent or a potent corticosteroid, however, it is the moderately or a potent corticosteroid is preferable, thus the corticosteroid can be Beta Methasone Dipropionate, Meometasone Furoate, Flucinolone Acetonide, Clobetasol, Triamconolone Acetonide. Keratolytic such as Salicylic Acid used in treatment of Psoriasis can also be included in the composition.

Method of Application—The topical composition can be applied on the areas requiring treatment can be made by fingers, glass rod, brush etc. However an applicator which is most suitable is in form of a nib adapted to a glass holder or a or a marker pen made out of amber colored glass, these nibs are of natural or synthetic fibers, porous plastic work on basis of capillary action Fiber tipped nibs of various sizes and shape are available the sizes vary 1-5 mm in various shapes Bullet/Chisel/pointed, These are available with Montana-Cans, Porous plastic nibs are available with Porex Technologies Malayisa. These nibs can be attached to suitable holder or a device having a reservoir, a marker pen with a amber colored glass reservoir is very suitable as the application can be a targeted fashion, however they can suitably be made custom designed to make it pharmaceutically acceptable i.e. the storage and delivery system should be as such to prevent any changes in the filled in material within the mentioned shelf life.

### SUMMARY AND EMBODIMENTS

According to one aspect of the invention a composition is provided comprising of at least one Topical photoactive agent along with a colorant as detectable marker so that when the topical composition is applied to an area over the skin, mucous membrane or at the muco cutaneous area it is visible and clearly differs from the rest of the area ware the application has not been made.

In another aspect of the invention steps of application of the topical photochemotherapy is provided for a subject in need of such treatment so that the area of application is targeted the application of the medicament is do not go beyond the area of the lesion and the area ware the application has been made is visible to eyes under sunlight/Ultra Violet Rays.

According to another aspect of the present invention there is provided a method of preparing a composition for topical photochemotherapy comprising of at least one photoactive agent along with a detectable marker.

According to yet another aspect of present invention there is provided steps of use of the topical photochemotherapeutic composition comprising of application of the topical followed by Ultra Violet Rays exposure to the area which is defined by the color of the detectable marker, the UVR used can be of any source which includes natural and/or artificial source, further the steps may involve the use of the composition along with Laser beams. The steps of usage may further involve application of the topical composition comprising of the Psoralen or it's derivative as photosensitizing agent along with the dye followed by Ultra Violet rays exposure which can immediate and/or a gap of few minutes to several hours as directed by the physician. Following the exposure the areas being treated ate wiped with aqua or another suitable solvent.

Therapeutic application of the invention can be in any dosage form suitable for topical use thus it will include Solution, Lotion, creams ointment. Depending on the pharmaceutical dosage form the formulation based on the invention may contain water, oily diluents, solvents, carrier, excepients, buffering agents, suspending agents emulsifier, emollients, humectants, stabilizing agents, dispersing agents, solubilizer, skin protectant, fragrance sunscreen agents textural modifier waterproofing agents and herbal extracts such as Aloe Vera extract.

In a preferred embodiment the composition is in a solution form, according to USP solutions are liquid preparation containing one or more ingredients dissolved i. e. molecularly dispersed in a suitable solvent or mixture of solvents thereof. The preferred solvents in this embodiment is Propylene glycol. Acetone, Ehtyl alcohol and purified water in which 8 methoxy psoralen (Methoxsalen) dye such as FCF Brilliant Blue is dissolved. Without limitation their solvents can also be used.

In a preferred embodiment the composition is in a solution form, according to USP solutions are liquid preparation containing one or more ingredients dissolved i. e. molecularly dispersed in a suitable solvent or mixture of solvents thereof. The preferred solvents in this embodiment is Propylene glycol. Acetone, alcohol and purified water in which 8 methoxy psoralen (Methoxsalen), Betamethasone Di propionate, dye such as FCF Brilliant Blue is dissolved.

In an another embodiment a lotion can be formulated comprising of the 8 Methoxy psoralen or any other Psoralen derivative along with a dye such as FCF Brilliant Blue which will impart a blue color, it can be a combination of color such as sunset yellow and Carmoisine in that case the lotion will be yellowish red color in color, other colorants can also be used provided they are visible on application over the area of treatment, beside the active ingredient and the dye other ingredients in this formulation can be Alcohol, Acetone, propylene glycol and purified water, other agents those may be present are methylcellulose, carboxy methyl cellulose or like. Other F. D. & C Regulatory approved colorants or combination thereof may also be used depending on the stability of the colorants. While choosing the color of the dye and/or pigment the complexion of the population of that geographical area may be taken in consideration so as to produce clear demarcation of the area to be treated. Without limitation any colorants as mentioned in patent application No.20100119561 can be used and by mention it is incorporated in entirety. The concentration of the dye should be minimum but adequate to produce clear demarcation.

In another preferred embodiment the colorants imparting color to the composition may vary according to the strength of the active ingredient Psoralen or it's derivative such as 8 Methoxy Psoralen being used, as the strengths of the active ingredient often depends on the area of treatment, Face and other exposed areas of the body require lesser strengths as compared to covered areas of the body.

In another embodiment the colorant being used is dispensed separate packing in dry form or in a solvent and other ingredients are packed in another packing this may be required when the shelf life of the colorant is short. In another embodiment a cream can be formulated containing the active ingredient along with a dye as marker i.e. when the cream is applied on the skin the area can identified, a cream is well known in the art are liquids or semisolid emulsion, either oil in water or water in oil, creams are washable containing an oil phase and a aqueous phase, the oil phase is usually petrolatum and fatty alcohol such as cetyl or stearyl alcohol. the emulsifier in cream preparation are generally anionic, cationic, non ionic or amphoteric surfactant. In yet another embodiment a dosage form may be formulated in form of an ointment, ointments are semisolid preparation typically based on petrolatum or other petrolatum derivatives. There are different kinds of ointment bases as will be appreciated by those skilled in the art and the best to be used while formulating will be one that provide optimum drug delivery. For a detailed description and understanding on ointment bases Remington's Pharmaceutical Sciences 16th Ed. is Editor Arthur OSOL 1980 pgs 1248-1253, in case of ointment suitable dye is incorporated along with the photoactive component Psoralen or it's derivative.

In an embodiment the dosage may be provided in a form of stick they are the dossier form prepared in a slender often cylindrical form as in chap stick or lipstick.

Other form of topical drug delivery system such as gels paste and films may also be formulated based on the invention containing the photoactive component Psoralen or it's derivative along with the a colorant and accordingly gelling agents, such as carbomer, film forming agents such as pyroxylin and in case of paste suitable base may be used.

The composition of this invention comprising of at least one photochemotherapeutic agent along with the detectable marker can incorporate other therapeutic agents those have been used in treatment of Vitiligo such as The topical Cortiticosteroid, by virtue of their action topical corticosteroid acts as an anti inflammatory agent and prevents the phototoxic effect of psoralen and at the same time exerts it therapeutic effect in treating vitiligo. Thus a combination of topical Psoralen along with topical corticosteroid is a rational combination to be used in therapy of Vitiligo, this combination can also be used in treatment of other photoresponsive drematoses such as but not limited to Psoriasis and Alopecia Areata, any of the topical corticosteroid listed above can be incorporated preferably the potent, mid potent corticosteroid such as Betamethasone Valerate, Fluticasone Propionate, Mometasone furoate, lesser potent corticosteroids may also be used particularly in children however it is the potent and mid potent topical steroids those preferable. Other topical agents those have been used in treatment of Vitiligo such as immune modular like Tacrolimus and Pimecrolimus, Placental extract, melagenina, Pseodocatalase Fibrocyte growth factor can also be incorporated in this invention comprising of topical Psoralen and a detectable marker. In reference to the use of tacrolimus which has been used both in localized as well as generalized vitiligo it is reported to have given best results on sun exposed areas and according to personal observation of N Ostovari, tacrolimus used as monotherapy without the Ultra violet exposure has little or no repigmenting potential (Dermatology Clinics Vol. 23, No. 2 April 2005 Pg 213, based on this reporting it can be inferred that Tacrolimus may have a photodyanamic property beside it's immunomodulatory property.

The composition of the present invention may further incorporate other active ingredients, used in treatment of Psoriasis, by definition active ingredients herein are agents those exert therapeutic effect or have been reported to be of therapeutic value, these may include topical Retinoids and its derivative Tazarotene, calcipotriol and coal tar.

In one of the embodiment the composition of the present invention may include one or more of biological additives such as botanicals and herbals, as used herein biological additives are those derived from natural source such as plants, animal, yeast, bacteria. examples of such biological include Aloe Vera, Henna, Turmeric, Coffee, Arnica, Gingko Biloba. References on botanicals can be found in related Pharmacopeia those include British Herbal Pharmacopeia, British herbal Association, Indian Ayurvedic pharmacopeia published Ayush Govt. Of india, Clinical Application of Ayurvedic & Chinese Herbs, D Reed, Shambala Boston

The composition of this invention can be given as monotherapy or it can be given along with other topical and/or systemic therapy.

The composition of present invention can be used in all subjects in need of such therapy without any consideration of age or sex, however, topical psoralen should be used cautiously children.

The composition may comprise of dyes those have photosensitive property such as Methylene blue, Toludine, rose Bengal, in this aspect of formulation the dyes act as both as a marker as well as a photosensitizer, further the composition may contain 1,3 dihydroxyacetone used in tanning of skin. The composition can include stains or dye having anti bacterial property beside coloring property such as Acraflavine, brilliant green gention violet.

The composition comprising Psoralen or its derivative thereof along with the colorant as the detectable marker may be packaged along with a separate container containing a washable solution which can be aqua and/or purified aqua, alcohol, acetone or a combination thereof or it can be another solvent dermatologically acceptable solution.

The composition when provided in a solution or lotion form can preferably be used with an applicator system so as to facilitate the application of the topical agent containing the photo active agent and the detectable marker in a targeted manner so that the medicament is applied only at the site of the lesion without going beyond the boundaries of the lesion and there are no trickling of the solution which often causes hyper-pigmentation or may even cause blistering reaction over the normal surrounding the lesion. Although application of the formulation can be made by fingers, however, as the lesions of Vitiligo are often very small with irregular borders an applicator system will facilitate better targeted application only confining to the lesion or lesions thereof, such applicator may preferably be in form of a brush, cotton tipped applicator, natural or man made fibre tipped applicator, porous plastic nibs, porous glass nibs, fiber glass nibs may be used as an applicator. The applicator may further have a reservoir and/or metered dose delivery system.

### FIGURES

FIG. 1 A depicts hyper pigmentation surrounding vitiligo lesion following topical Psoralen therapy

FIG. 1 B Erythematous photo toxic reaction involving the normal skin surrounding the vitiligo lesion following two application of 1.0% solution of 8 methoxypsoralen at alternate days.

FIG. 2 A depicts multiple vitiligo lesions of irregular shapes with the irregular borders, difficult for topical application with fingers as there is always a chance of crossing the boundaries and spreading of the topical composition with presently available formulation.

FIG. 2-B Shows use of a fiber tipped nib attached to a holder soaked in the composition being applied on a volunteer having Vitiligo.

FIG. 2 C shows the application of the colored composition of the without crossing the borders

FIG. 3-A Shows a very small lesion of vitiligo on foot

FIG. 3-B shows the application of the colored composition without crossing the borders.

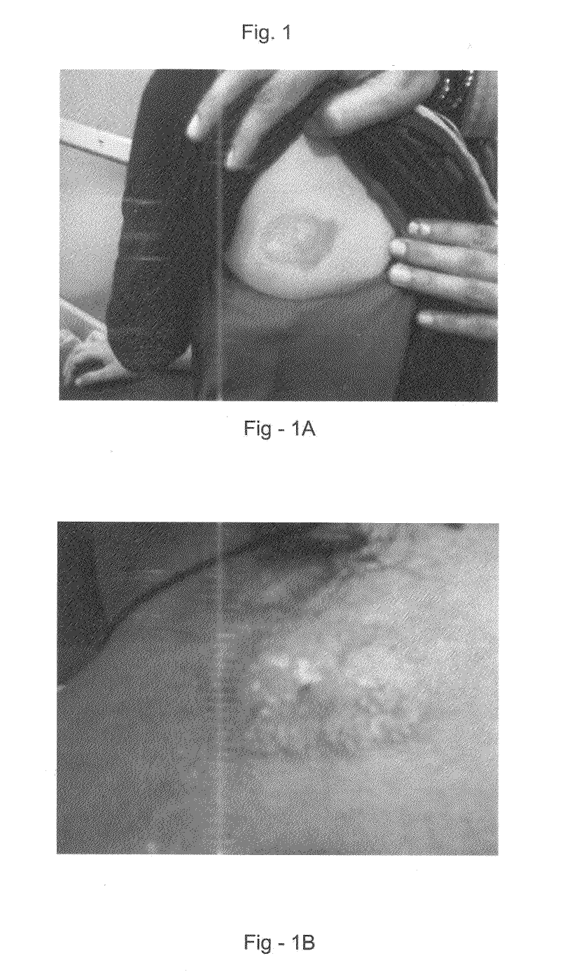

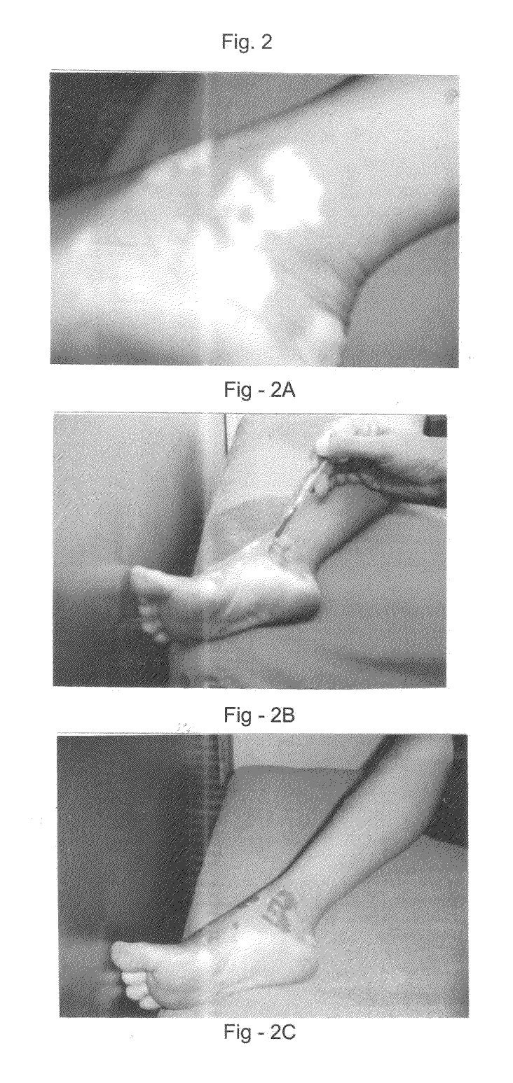

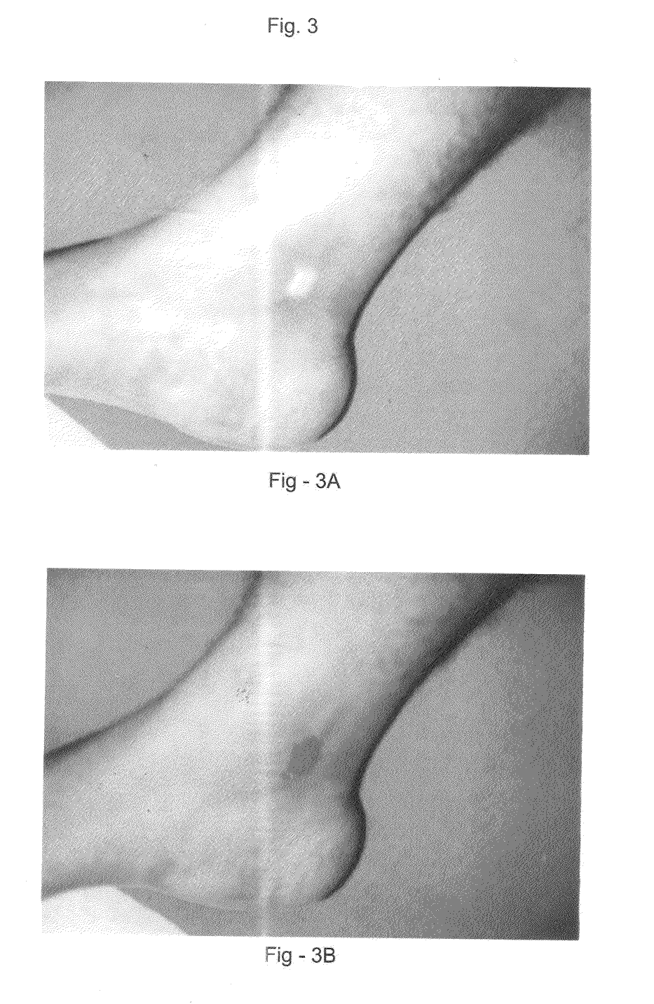

### EXAMPLE-I

8 Methoxy psoralen—1.00 gm, Propylene Glycol—13.40 ml, Acetone −13.40 ml FCF Brilliant Blue—q.s. Alcohol 70% to make 100 ml

N. B. The Dye FCF Brilliant Blue Supra is available as synthetic food color IDACOL (Manufactured by Roha Dye chem.)

70% Alcohol is prepared by diluting Absolute Alcohol 99.99% (Analytical reagent Changshu Yangyuan Chemical, China) by volume according to Ramington's Pharmaceutical Sceinces 1980

Methoxsalen availed from M/s Jae radhey Sales Ahmedabad

Method of preparation—Accurately weigh or measure each ingredient, dissolve Methoxy psoralen and the dye in Acetone, Propylene glycol and about 65 ml of 70% alcohol. Add sufficient 70% alcohol to make final volume and mix well.

### EXAMPLE-II

8 Methoxy psoralen—1.00 gm, Beta Methasone Dipropionate—0.025 gm, Propylene Glycol—13.40 ml, Acetone—13.40 ml Orange Dye—1.00 gm., Alcohol 70% to make—100 .ml

(70% Alcohol prepared by diluting Absolute Alcohol 99.99 (Analytical reagent Changshu Yangyuan Chemical, China) by volume according to Ramington's Pharmaceutical Sceinces 1980

N.B.—The Food dye (orange red color) used here is combination of Sunset Yellow and Carmosine a food dye available under the trade name of Anuja in India it is a sodium salt of the dye and the Dye content is 30.20%

Betamethasone Dipropinate Granth Pharmaceuticals (P) Ltd through Mehta Associates, Mumbai

Method of preparation—Accurately weigh or measure each ingredient, dissolve Methoxy psoralen and Beta Methasone di propionate and the dye in Acetone, Propylene glycol and about 65 ml of 70% alcohol. Add sufficient 70% alcohol to final volume and mix well.

The references on patents, articles, monographs, chapters from the books appearing herein above are incorporated in entirety by mention.

It will be appreciated by those skilled in the art that many modifications, addition, alteration, substitution can be made without departing from the true spirit of invention. The accompanying claims are thus to be understood to include what has been specifically described herein above and also what can be obviously substituted, conceptually equivalent and various modifications with out departing from the true spirit of invention.

### REFERENCES

IADVL Consensus Guidelines 2008—Medical Management of Vitiligo Published by Indian Assodiation of Dermatologists, Venereologists & Leprologists Pg-8-23 Kandil E, Treatment of localized Vitiligo with intralesional Triamcolone Acetonide, Dermatologica !970, 140;195

Dermatology Clinics Vol 23 No. 2 April 2005 Jean-Paul Thierry Passeron—Melanin Pigmentory Disorder Treatment Update

Krishns vamshi alam, gannu Parveen kumar Colorants—The cosmetics for the pharmaceutical dosahe Form—International journal of Pharmacy and Pharmaceutical Science Vol. 3, Suppl. 3 2011.

Percy Lehmann—Photodermatosis Braun Falco's Dermatology Chapter 41

Dr. Antonio Salafia—VITILIGO—Leukoderma—Textbook and Atlas 2010 Website of FDA on approved colorants inventories visited on 22, Apr. 2013

I/We claim:

1. A topical composition comprising of at least one photoactive agent and a colorant which can be a tint, stain, paint, pigment dye or it's Lake, wherein when the composition is topically applied the presence of the photoactive agent is indicated by the color imparted by the colorant which serves as a detectable marker.

2. Composition of claim 1 wherein the Photoactive agent is Psoralen or a Psoralen based compound.

3. Composition of claim 1 wherein the photoactive agent is natural or synthetic derivative of Psoralen, preferably it is 4, 5, 8, Trimethyl psoralen, 5 methoxy psoralen or 8 methoxy psoralen more preferably it is 8 Methoxypsoralen (Methoxsalen).

4. The composition of claim 3 wherein the dose of 8 Methoxy psoralen is between 0.001 to 10 percent, preferably it is between 0.01-2, percent more preferably it is between 0.1-2.0% still more preferably it is between 0.75-1.0%

5. The composition of claim 1 wherein the colorant or a mixture of colorants thereof being used is a tint, stain, paint, dye or a pigment, preferably the colorant being used is a dye or its lake, more preferably it is a FDA approved colorant or approved for use in Food, Drug and/or Cosmetics in a country of it's produce.

6. The composition of claim 5 wherein the colorant being used is in sufficient quantity/concentration so that it is visible in presence of light and/or in presence of Ultra violet Rays and clear demarcation can be made wherein topical application has been made.

7. The composition of claim 1 wherein the colorant being used has: (a) Property of Photosensitization in addition to it's property of imparting color such as Bengal rose, methylene blue.
(b) Antibacterial property beside the property of imparting the coloring effect to the composition, the colorant can be a stain or dye or a combination thereof such as acraflavine, brilliant green, gentian violet or any other having such property

8. The composition of claim 1 wherein the composition incorporates one or more of the active ingredients selected from the group consisting of anti inflammatory, immunomodulator, keratolytic, sun screen or sun tanning agents.

9. The composition of claim 8 wherein the anti inflammatory agent is corticosteroid selected from mild, moderate or potent, preferably it is a of mid potent or a potent corticosteroid more preferably it is Betamethasone Dipropionate, Mometasone furoate, Fluticasone propionate or Clobetasol.

10. The composition of claim 8, wherein it comprises of a topical immunomodulator such as Tacrolilus, pimecrolimus, Calcipotril, or another active ingredient used in treatment of Vitiligo such as Fibroblast growth factor, Melagenina and/or Placental extract, preferably it is Tacrolimus

11. The composition of claims 1 and 8. Wherein the dosage form is a solution, lotion, cream, ointment or gel, preferably it is in a solution form.

12. The composition of claims 1 and 8 having photoactive property is being used for treatment of various dermatological conditions including but not limited to Vitiligo, Psoriasis and alopecia Areata.

13. Composition of claims 1 and 8 wherein depending on the dosage form of the composition include one or more of ingredients such as aqueous or oily diluents, carrier, buffering agents, emulsifier, emollient, thickening agents, gelling agents, fragrance, surfectants, skin protectant, preservatives, excipients

14. Composition of claim 1 or 8 wherein the composition further incorporates agents derived from plants and herbs such as Henna, Aloe vera, Tea Tree oil other Pharmaceutically and dermatologically acceptable herbs as mentioned in Indian Ayurvedic Pharmacopaeia published by Ayush A government of India publication.

15. The method of use of the composition of claim 1 or 8 involve the use of light, the source of light may be natural sunlight or Ultra Violet rays which includes UVA and/or UVB, the composition can also be used along with laser beams preferably the source of light is UVA or UVB more preferably it is UVA component be used when UVR is used.

16. Composition of claim 1 and 8 wherein the composition is dispensed in an applicator system with or without a reservoir, suitably be adapted to a nib which facilitates application of the composition so that the area of application do not cross the boundaries of the lesion and there is no trickling of the composition when applied, preferably the nib is of synthetic or natural fiber or a combination thereof, porous plastic, wood or glass.

17. Composition of claim 16 wherein the colorant being used is impregnated in the nib and other ingredients of the composition is separately packaged.

18. Composition of claim 16 wherein the colorant being used is separately dispensed in dry or liquid form and the photoactive ingredient or other ingredients are dispensed in separate packing

19. The composition of claim 1 and 8 is dispensed along with aqua, solvent or a mixture thereof in a separate container for wiping of the composition from the area of it's application.

20. Composition of claims 1 and 8 is dispensed in a dosage form of Stick for topical application.
